(19) United States
(12) Patent Application Publication
(10) Pub. No.: US 2025/0049513 A1
(43) Pub. Date: Feb. 13, 2025

(54) NAVIGATION ADAPTER SLEEVE FOR ENT INSTRUMENT

(71) Applicant: Acclarent, Inc., Irvine, CA (US)

(72) Inventors: Gigi KOE, Anaheim, CA (US); Alex GONZALEZ, Lake Forest, CA (US); Henry F. SALAZAR, Pico Rivera, CA (US); Raymond Yue-sing TANG, Rosemead, CA (US)

(21) Appl. No.: 18/798,677

(22) Filed: Aug. 8, 2024

Related U.S. Application Data
(60) Provisional application No. 63/531,683, filed on Aug. 9, 2023.

(51) Int. Cl. A61B 34/20; A61B 17/00; A61B 17/24; A61B 17/32

(52) U.S. Cl. CPC A61B 34/20; A61B 17/24; A61B 17/32002; A61B 2017/00486; A61B 2034/2072

(57) ABSTRACT

An apparatus for use with an instrument includes a hub, a shaft assembly, and a navigation sensor. The hub is configured to engage a portion of the instrument, in only a predetermined angular orientation with respect to an axis of the instrument. The shaft assembly extends from the hub and defines a hollow interior and an open distal end. The hollow interior is configured to receive a tubular portion of the instrument. The navigation sensor can be disposed proximate the open distal end of the shaft assembly. The navigation sensor can be configured to generate a signal indicating a portion of at least a portion of the shaft assembly in three-dimensional space.

### CROSS-REFERENCE TO RELATED APPLICATIONS

This application claims the benefit of U.S. Provisional Application No. 63/531,683, filed Aug. 9, 2023, the entirety of which is incorporated herein by reference.

### BACKGROUND

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. An example of an electromagnetic IGS navigation system that may be used in IGS procedures is the CARTO® 3 System by Biosense-Webster, Inc., of Irvine, California. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, some instruments can include sensors (e.g., electromagnetic coils that emit electromagnetic fields and/or are responsive to externally generated electromagnetic fields), which can be used to perform the procedure while the sensors send data to the computer indicating the current position of each sensor-equipped instrument. The computer correlates the data it receives from the sensors with the digital map that was created from the preoperative tomographic scan. The tomographic scan images are displayed on a video monitor along with an indicator (e.g., crosshairs, an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

In some instances, it may be desirable to guide an instrument using IGS techniques described above. However, certain IGS navigation systems may be equipped for use with specific instruments and certain instruments may be equipped for use with specific IGS navigation systems or no IGS navigation system at all. Thus, it may be desirable to provide an easily implemented and cost-effective means to incorporate compatibility with a given IGS navigation system into a variety of instruments that otherwise lack position sensing features. While several systems and methods have been made and used in connection with IGS navigation stems, it is believed that no one prior to the inventors has made or used the inventions described in the appended claims.

### BRIEF DESCRIPTION OF THE DRAWINGS

The drawings and detailed description that follow are intended to be merely illustrative and are not intended to limit the scope of the inventions as contemplated by the inventors.

FIG. 1 depicts a schematic view of an example of a surgery navigation system being used on a patient seated in an example of a medical procedure chair, according to some embodiments.

FIG. 2 depicts a perspective view of an example of a tissue shaving instrument, according to some embodiments.

FIG. 3 depicts a perspective view of an example of an adapter sheath for use in combination with the surgery navigation system of FIG. 1 and the instrument of FIG. 2, according to some embodiments.

FIG. 4A depicts a perspective view of the tissue shaving instrument of FIG. 2 with the adapter sheath of FIG. 3 being inserted onto the tissue shaving instrument, according to some embodiments.

FIG. 4B depicts another perspective view of the tissue shaving instrument of FIG. 2, the adapter sheath of FIG. 3 being secured to the tissue shaving instrument, according to some embodiments.

### DETAILED DESCRIPTION

The following description of certain examples of the inventions should not be used to limit the scope of the present inventions. Other examples, features, aspects, embodiments, and advantages of the inventions will become apparent to those skilled in the art from the following description, which is by way of illustration, one of the best modes contemplated for carrying out the inventions. As will be realized, the inventions are capable of other different and obvious aspects, all without departing from the inventions. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. Example of an Image Guided Surgery Navigation System

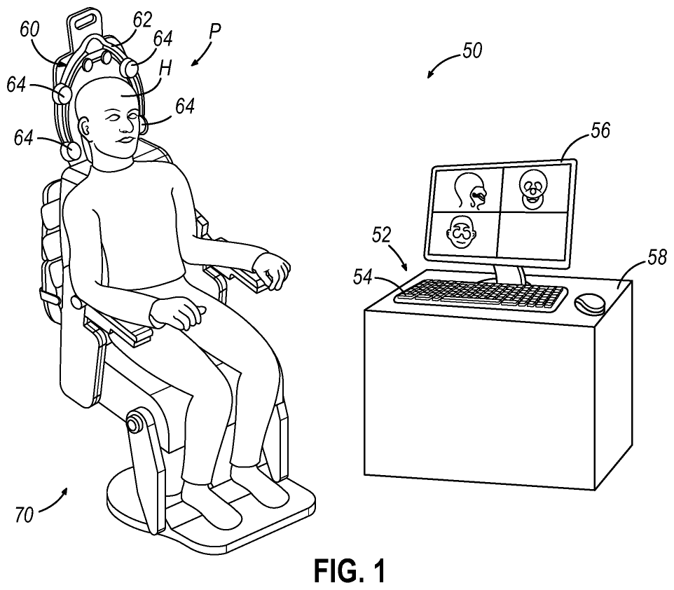

When performing a medical procedure within a head of a patient (P), it may be desirable to have information regarding the position of an instrument within the head (H) of the patient (P), particularly when the instrument is in a location where it is difficult or impossible to obtain an endoscopic view of a working element of the instrument within the head of the patient (P). FIG. 1 shows an example of an IGS navigation system 50 enabling a medical procedure to be performed within a head (H) of a patient (P) using image guidance. In addition to or in lieu of having the components and operability described herein, the IGS navigation system 50 may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010; and/or U.S. Pat. No. 11,065,061, entitled “Systems and Methods for Performing Image Guided Procedures within the Ear, Nose, Throat and Paranasal Sinuses,” issued Jul. 20, 2021. The disclosures of U.S. Pat. No. 7,720,521, entitled “Methods and Devices for Performing Procedures within the Ear, Nose, Throat and Paranasal Sinuses,” issued May 18, 2010, and U.S. Pat. No. 11,065,061, entitled “Systems and Methods for Performing Image Guided Procedures within the Ear, Nose, Throat and Paranasal Sinuses,” issued Jul. 20, 2021, are incorporated by reference herein, in their entirety.

The IGS navigation system 50 of the present example comprises a field generator assembly 60, which comprises a set of magnetic field generators 64 that are integrated into a horseshoe-shaped frame 62. The field generators 64 are operable to generate alternating magnetic fields of different frequencies around the head (H) of the patient (P). An instrument may be inserted into the head (H) of the patient (P). Such an instrument may include one or more position sensors as described in greater detail below. In the present example, the frame 62 is mounted to a chair 70, with the patient (P) being seated in the chair 70 such that the frame 62 is located adjacent to the head (H) of the patient (P). By way of example only, the chair 70 and/or the field generator assembly 60 may be configured and operable in accordance with at least some of the teachings of U.S. Pat. No. 10,561,370, entitled “Apparatus to Secure Field Generating Device to Chair,” issued Feb. 18, 2020. The disclosure of U.S. Pat. No. 10,561,370, entitled “Apparatus to Secure Field Generating Device to Chair,” issued Feb. 18, 2020, is incorporated by reference herein, in its entirety. In some other variations, the patient (P) lies on a table, and the field generator assembly 60 is positioned on or near the table.

The IGS navigation system 50 of the present example further comprises a processor 52, which controls the field generators 64 and other elements of the IGS navigation system 50. For instance, the processor 52 is operable to drive the field generators 64 to generate alternating electromagnetic fields; and process signals from the instrument to determine the location of a navigation sensor or position sensor in the instrument within the head (H) of the patient (P). The processor 52 comprises a processing unit (e.g., a set of electronic circuits arranged to evaluate and execute software instructions using combinational logic circuitry or other similar circuitry) communicating with one or more memories. The processor 52 of the present example is mounted in a console 58, which comprises operating controls 54 that include a keypad and/or a pointing device such as a mouse or trackball. A physician uses the operating controls 54 to interact with the processor 52 while performing the surgical procedure.

While not shown, the instrument may include a navigation sensor or position sensor that is responsive to positioning within the alternating magnetic fields generated by the field generators 64. A coupling unit (not shown) may be secured to the proximal end of the instrument and may be configured to provide communication of data and other signals between the console 58 and the instrument. The coupling unit may provide wired or wireless communication of data and other signals.

In some versions, the navigation sensor or position sensor of the instrument may comprise at least one coil at or near the distal end of the instrument. When such a coil is positioned within an alternating electromagnetic field generated by the field generators 64, the alternating magnetic field may generate electrical current in the coil, and this electrical current may be communicated along the electrical conduit(s) in the instrument and further to the processor 52 via the coupling unit. This phenomenon may enable the IGS navigation system 50 to determine the location of the distal end of the instrument within a three-dimensional space (i.e., within the head (H) of the patient (P), etc.). To accomplish this, the processor 52 executes an algorithm to calculate location coordinates of the distal end of the instrument from the position related signals of the coil(s) in the instrument. Thus, a navigation sensor may serve as a position sensor by generating signals indicating the real-time position of the sensor within three-dimensional space.

The processor 52 uses software stored in a memory of the processor 52 to calibrate and operate the IGS navigation system 50. Such operation includes driving the field generators 64, processing data from the instrument, processing data from the operating controls 54, and driving a display screen 56. In some implementations, operation may also include monitoring and enforcement of one or more safety features or functions of the IGS navigation system 50. The processor 52 is further operable to provide video in real time via the display screen 56, showing the position of the distal end of the instrument in relation to a video camera image of the patient's head (H), a CT scan image of the patient's head (H), and/or a computer-generated three-dimensional model of the anatomy within and adjacent to the patient's nasal cavity. The display screen 56 may display such images simultaneously and/or superimposed on each other during the surgical procedure. Such displayed images may also include graphical representations of instruments that are inserted in the patient's head (H), such that the operator may view the virtual rendering of the instrument at its actual location in real time. By way of example only, the display screen 56 may provide images in accordance with at least some of the teachings of U.S. Pat. No. 10,463,242, entitled “Guidewire Navigation for Sinuplasty,” issued Nov. 5, 2019. The disclosure of U.S. Pat. No. 10,463,242, entitled “Guidewire Navigation for Sinuplasty,” issued Nov. 5, 2019, is incorporated by reference herein, in its entirety. In the event that the operator is also using an endoscope, the endoscopic image may also be provided on the display screen 56.

The images provided through the display screen 56 may help guide the operator in maneuvering and otherwise manipulating instruments within the patient's head (H). It should also be understood that other components of a surgical instrument and other kinds of surgical instruments, as described below, may incorporate a navigation sensor like the navigation sensor described above.

### II. Example of a Tissue Shaving Instrument

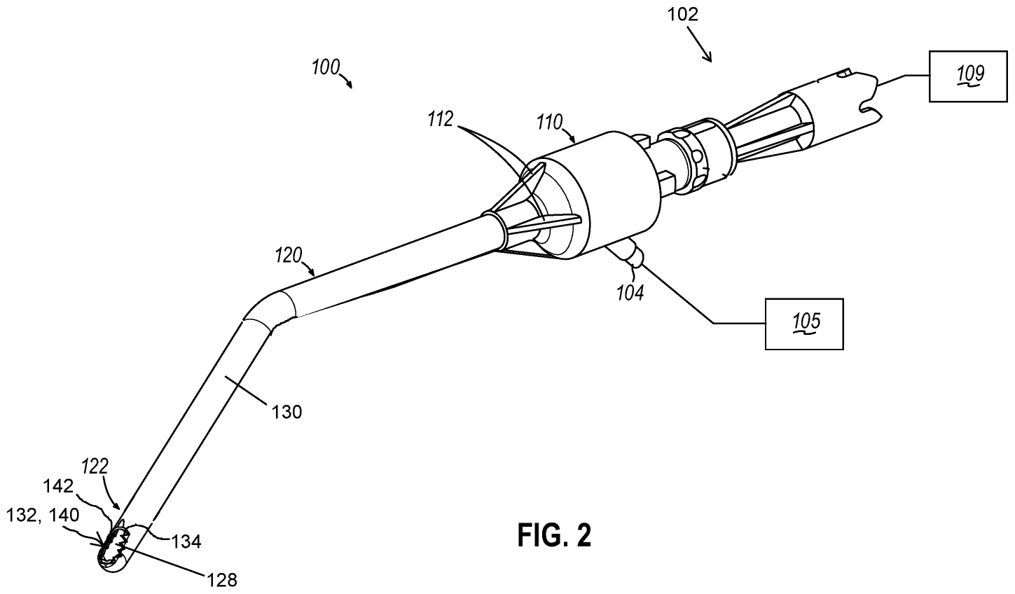

FIG. 2 shows an example of a tissue shaving instrument 100, which is configured for use in a surgical procedure (e.g., a FESS procedure, a turbinate reduction procedure, etc.) where bone and/or soft tissue is removed from within the ear, nose, or throat of a patient. As will be described in greater detail below, it may be desirable to adapt the tissue shaving instrument 100 or other instruments for use with the IGS navigation system 50 because the tissue shaving instrument 100 itself may lack position sensors configured for use with the IGS navigation system 50. While the example described below is in the context of the tissue shaving instrument 100, the teachings below may be readily applied in the contexts of other instruments, including but not limited to bur instruments, drill instruments, suction instruments, and other instruments as will be apparent to those skilled in the art in view of the teachings herein.

The tissue shaving instrument 100 of this example includes a proximal hub assembly 102, a distal hub 110, and a shaft assembly 120. The distal hub 110 is configured to be fixedly secured to a body (not shown), while the proximal hub assembly 102 is configured to be inserted into the body. By way of example only, the body may comprise a handle that is configured for grasping by an operator. The distal hub 110 includes a fluid port 104 that is configured to couple with a fluid source 105. In some examples, the fluid port 104 and the fluid source 105 are configured to apply suction to draw tissue and fluids during operation of the tissue shaving instrument 100. In addition, or in the alternative, the fluid port 104 and the fluid source 105 may be configured to communicate fluids to irrigate patient anatomy during operation of the tissue shaving instrument 100.

The proximal hub assembly 102 is rotatable relative to the distal hub 110. In the present example, the proximal hub assembly 102 is coupled with a multi-function control unit 109. The multi-function control unit 109 is configured to communicate power, signals, and/or fluid for the operation of the tissue shaving instrument 100. For instance, in some examples, the multi-function control unit 109 is configured to provide power to a motor (not shown) that is disposed in in the same body to which the distal hub 110 is coupled; and in which the proximal hub assembly 102 is disposed. Such a motor may be operable to drive one or more components of the proximal hub assembly 102, to thereby drive rotation of a cutting shaft 128 of the shaft assembly 120 as will be described in greater detail below. As another variation, the motor may be positioned remotely from the body to which the distal hub 110 is coupled, and in which the proximal hub assembly 102 is disposed, and a rotary drive cable may be coupled with the proximal hub assembly 102 to communicate rotary motion from the remote motor to the tissue shaving instrument 100. In some versions, the multi-function control unit 109 is also configured to provide suction to the tissue shaving instrument 100. Such suction may be used to draw tissue or fluids during operation of the tissue shaving instrument 100. In some versions, the multi-function control unit 109 is configured to activate the fluid source 105.

The distal hub 110 is configured to couple with a body (e.g., handle) as described above; and in some versions this coupling is removable such that the distal hub 110 may be decoupled from the body after being coupled with the body. The hub 110 defines a generally cylindrical shape with the fluid port 104 extending from a portion thereof. As noted herein, the fluid port 104 may be in communication with a portion of the shaft assembly 120 via the distal hub 110. The distal hub 110 further includes a plurality of ribs 112 extending from a distal portion of the distal hub 110. The ribs 112 may be configured to provide structural support between the distal hub 110 and the shaft assembly 120. In addition, as will be described in greater detail below, the ribs 112 may be used as location features to index the distal hub 110 relative to other features of the tissue shaving instrument 100. Additionally, as will be described in greater detail below, the ribs 112 may be used to index other accessory components relative to the distal hub 110 and/or the tissue shaving instrument 100 in some examples. Although the ribs 112 are shown as triangular fin-shaped structures in the present example, it should be understood that other geometries may be used in other examples.

The shaft assembly 120 extends distally from the distal hub 110 and the proximal hub assembly 102. The shaft assembly 120 includes an inner shaft 128 and an outer shaft 130. Both the inner shaft 128 and the outer shaft 130 are coaxial with each other and define a substantially similar shape with a straight portion and a bent portion. It should be understood that the bent portion is entirely optional and may be omitted in some examples, such that the shaft assembly 120 may be entirely straight in some variations.

The outer shaft 130 generally extends from the hub 110, while the inner shaft 128 extends from the proximal hub assembly 102. In this configuration, fluids may be communicated via the hub 110 and the fluid port 104 through the interior of the outer shaft 130, along a radially extending gap defined between the inner shaft 128 and the outer shaft 130. Such fluids may be communicated to apply irrigation or suction during use of the tissue shaving instrument 100. Additionally, both the hub 110 and the outer shaft 130 may be removably secured to the proximal hub assembly 102 and or the inner shaft 128 such that the hub 110 and the outer shaft 130 may be removable. Thus, it should be understood that the hub 110 and or the outer shaft 130 are entirely optional and may be omitted in some examples.

An end effector 122 is formed at the distal end of the shaft assembly 120. The end effector 122 includes a first transverse opening 132 formed at the distal end of the outer shaft 130 and a second transverse opening 140 formed at the distal end of the inner shaft 128. The transverse openings 132, 140 share a common longitudinal position with each other. The transverse openings 132, 140 may alternate between being angularly aligned with each other and being angularly offset from each other as the inner shaft 128 is rotated relative to the outer shaft 130 by the proximal hub assembly 102. The first transverse opening 132 is bounded by a first edge 134 while the second transverse opening 140 is bounded by a second edge 142. The edges 134, 142 cooperate to shear tissue that protrudes into the openings 132, 140 as the inner shaft 128 is rotated relative to the outer shaft 130. One or both of the edges 134, 142 may include serrations or may be otherwise sharp to promote cutting of tissue by the end effector 122. During such rotation, suction is applied via a lumen defined by the inner shaft 128 to draw tissue into the transverse opening 140, the edges 134, 142 shear the tissue, and the sheared tissue is drawn proximally through the lumen of the inner shaft 128 under the influence of the suction.

### III. Example of a Navigation Adapter for Providing Navigation to an Instrument

Those skilled in the art will understand that it may be beneficial to have data from one or more navigation sensors indicating the real-time location of the end effector 122 within the patient (P) during operation of the tissue shaving instrument 100. This may be particularly desirable in scenarios where the end effector 122 is used within the head of the patient, where endoscopic visualization may be difficult, and where sensitive anatomical features present very little margin for error in positioning of the end effector 122. To that end, it may be desirable to provide an adapter or other component having one or more integral navigation sensors. Such an adapter may be readily coupled with an instrument, such as the tissue shaving instrument 100 described above, that otherwise lacks any navigation sensors. In such a scenario, the adapter may impart navigation capabilities to the instrument.

Similarly, it may be desirable to use an adapter having one or more integral navigation sensors in combination with an instrument that already has one or more navigation sensors, where the position data from the one or more navigation sensors of the adapter may supplement the position data from the one or more navigation sensors of the instrument. In such a scenario, the adapter may enhance navigation capabilities of the instrument. In either of the above scenarios, the adapter may be configured to avoid adding bulk to the instrument; and to be easily assembled with the instrument in the surgical field or operating room. Moreover, the adapter may be configured to accommodate different kinds of instruments and/or instruments otherwise having different outer diameters, such that the adapter need not necessarily be limited in its compatibility to just one single instrument size or type.

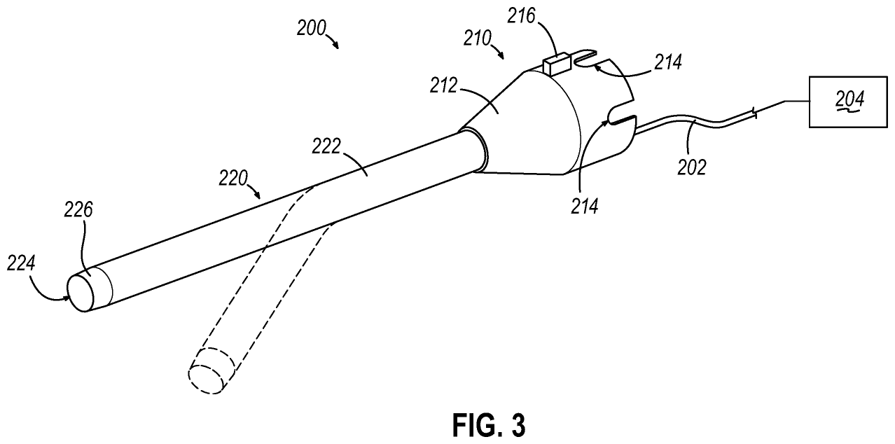

FIG. 3 shows an example of an adapter, that may provide the above-described benefits and functionality, in the form of a flexible adapter 200. In the present example, the flexible adapter 200 is configured to couple with the tissue shaving instrument 100. As will be described in greater detail below, adapters similar to the flexible adapter 200 of the present example may be used in combination with instruments similar to the tissue shaving instrument 100 to adapt such instruments for navigation using navigation systems such as the IGS navigation system 50. In particular, existing instruments may lack features such as one or more integral navigation sensors to facilitate navigation of such instruments via systems such as the IGS navigation system 50. Therefore, it may be desirable to add navigation capabilities to such instruments via components such as adapters. Such a combination may be desirable to facilitate the use of certain virtual reality features and/or advanced mapping features. Additionally, such a combination may be desirable to facilitate real-time overlay of positional data on images, providing true anatomical measurements that may improve workflows and/or procedure outcomes. Although certain combinations of adapters and instruments are described herein, it should be understood that such combinations may be combined with other adapters and/or instruments to promote use of the IGS navigation system 50 with multiple instruments used in a given procedure.

The flexible adapter 200 of the present example includes a hub 210, a shaft assembly 220 extending distally from hub 210, a cable 202, and a connector 204. As will be described in greater detail below, the flexible adapter 200 is generally configured to mate with the tissue shaving instrument 100 to add, or otherwise supplement, navigation capabilities with respect to tissue shaving instrument 100. Thus, flexible adapter 200 includes one or more navigation sensors configured for use in combination with IGS navigation system 50. As will also be described in greater detail below, such navigation sensors are configured to locate one or more geometric features of the flexible adapter 200 in three-dimensional space using the IGS navigation system 50. Such geometric features of the flexible adapter 200 can then be correlated with one or more features of the tissue shaving instrument 100 (e.g., the end effector 122, and more particularly the first transverse opening 132, etc.) to likewise locate one or more features of tissue shaving instrument 100 in three-dimensional space.

The shaft assembly 220 includes a sleeve 222 extending distally from hub 210. The sleeve 222 terminates an open distal end 224. In the present example, the sleeve 222 defines a length less than the length of a portion of the shaft assembly 120 of the tissue shaving instrument 100 such as the inner shaft 128. As will be described in greater detail below, such a differential length permits the end effector 122 to protrude from the open distal end 224. Although the shaft assembly 220 of the present example is configured with only the sleeve 222, it should be understood that in other examples, the shaft assembly 220 may optionally include multiple sleeves layered together.

The sleeve 222 is generally of a flexible configuration. As can be seen in FIG. 3, such flexibility is configured to permit bending of the sleeve 222 to accommodate a variety of shapes such as a bent shape defined by the shaft assembly 120 of the tissue shaving instrument 100; or a variation of the shaft assembly 120 having a straight configuration. At least some flexibility of the sleeve 222 may additionally be desirable to promote use of the sleeve 222 with other instruments having differently sized shafts similar to the shaft assembly 120. Flexibility of the sleeve 222 may additionally be desirable to provide a compression fit between the sleeve 222 and the shaft assembly 120 of the tissue shaving instrument 100. As will be described in greater detail below, such a compression fit may be configured to provide sealing between the sleeve 222 and the shaft assembly 120, thereby preventing fluid ingress at the interface thereof.

The shaft assembly 220 further includes one or more navigation sensors 226. Incorporation of one or more navigation sensors 226 may be desirable to add navigation functionality to the tissue shaving instrument 100 via the flexible adapter 200 and the IGS navigation system 50. In the present example, at least one navigation sensor 226 is positioned proximate the open distal end 224. Such a positioning may be desirable to facilitate localization of the end effector 122 due to the proximate relationship between the open distal end 224, the navigation sensor 226, and the end effector 122. Although not shown, it should be understood that in other examples, additional navigation sensors 226 may be included at various positions along the length of the sleeve 222. Such additional navigation sensors 226 may be desirable to promote enhanced localization of the shaft assembly 220 and/or other components of the sleeve 200. In addition, or in the alternative, one or more navigation sensors 226 may be incorporated into other structures of the flexible adapter 200 such as the hub 210.

The navigation sensors 226 are generally coupled to at least a portion of the sleeve 222. For instance, in some examples, the navigation sensors 226 may be configured as stickers sensors that may be adhered to the interior or exterior of the sleeve 222. In multi-sleeve configurations of the shaft assembly 220, the navigation sensors 226 may be layered between two sleeves and secured via adhesive, friction, or both. In yet other examples, the navigation sensors 226 may be integral with a portion of the sleeve 222 with a portion of each navigation sensor 226 being embedded within the structure of the sleeve 222 (e.g., by being formed as an integral feature of a flex circuit with the sleeve 222). Still other means of coupling the navigation sensors 226 to the sleeve 222 or other components of the shaft assembly 220 will be apparent to those of ordinary skill in the art in view of the teachings herein.

The navigation sensors 226 are in communication with the connector 204 via the cable 202. As best seen in FIG. 1, such communication is configured to facilitate communication of the navigation sensors 226 with console of the IGS navigation system 50 via the connector 204. Although not shown, it should be understood that such communication may be facilitated by a plurality of electrical communication paths extending through the sleeve 222 to the open distal end 224. Such electrical communication paths may take on a variety of forms. For instance, in some examples, such electrical communication paths may be configured as traces on a flexible printed circuit board or flex circuit attached to (e.g., on the inner diameter or outer diameter) or embedded within a portion of the sleeve 222. Such flexible printed circuit boards may additionally include one or more leads to facilitate electrical communication between the wires 202 and/or the connector 204. In other examples, such electrical communication paths may be configured as a plurality of wires extending through the sleeve 222. Such wires may likewise be attached to or embedded within a portion of the sleeve 222. Alternatively, in some examples, the sleeve 222 may be configured to define one or more dedicated lumens configured to receive one or more of such wires. Of course, various other means of facilitating electrical communication paths through the sleeve 222 will be apparent to those of ordinary skill in the art in view of the teachings herein.

The hub 210 is disposed on a proximal end of the shaft assembly 220 and is generally configured to facilitate various components of the tissue shaving instrument 100 to interface with the shaft assembly 220. The hub 210 includes a body 212, which defines one or more receiving features 214 and an alignment protrusion 216. As will be described in greater detail below, the receiving features 214 and the alignment protrusion 216 are configured to engage the hub 110 of the tissue shaving instrument 100 cooperatively to provide a poka-yoke feature. In some embodiments, such a feature may permit coupling of the hub 210 of the flexible adapter 200 to the hub 110 of the tissue shaving instrument 100 in one or more specific or predetermined ways. In some embodiments, the hub 210 of the flexible adapter 200 may be coupled to the hub 110 of the tissue shaving instrument 100 in only one or more relative positions or orientations. The positions or angular orientations may be determined, set, or selected prior to assembly of the hub 210 and flexible adapter 200. In some embodiments, the flexible adapter 200 may be coupled with the tissue shaving instrument 100 at only one, predetermined angular relationship with the tissue shaving instrument 100. Such a poka-yoke feature may be desirable in some examples to consistently promote predetermined angular alignment between one or more navigation sensors 226 and certain features of the end effector 122, such as the first transverse opening 132.

The receiving features 214 in the present example include a plurality of slots, channels, or openings extending distally into the surface of the body 212 from a proximal end of the hub 210. Each receiving feature 214 is generally configured to be complementary to a corresponding rib 112 of the hub 110 of the tissue shaving instrument 100. Thus, each receiving feature 214 is generally configured to receive a corresponding rib 112. Thus, it should be understood that in examples where the particular configuration of ribs 112 are varied, the particular configuration of each receiving feature 214 may likewise be varied.

The shape of each receiving feature 214 is generally complementary to the shape of each rib 112. Thus, in the present example, the slotted configuration of each receiving feature 214 corresponds to the fin-shaped structure of each rib 112. In some examples, each receiving feature 214 and rib 112 may be substantially similar to each other receiving feature 214 and rib 112. In other examples, the configuration of an individual receiving feature 214 and corresponding rib 112 may be varied to provide a keyed relationship with respect to receiving features 214 and ribs 112. For instance, in one example, one receiving feature 214 and rib 112 pair may have a unique shape or size relative to the other receiving features 214 and ribs 112. Consequently, in some embodiments, the unique receiving feature 214 and rib 112 pair may only mate with each other and not with other receiving features 214 and ribs 112, thereby providing a keyed relationship with respect to the hubs 110, 210 where the hubs 110, 210 may only be engaged in a single position relative to each other. Of course, other suitable configurations to provide such a keyed relationship with respect to the hubs 110, 210 will be apparent to those of ordinary skill in the art in view of the teachings herein.

The alignment protrusion 216 extends outwardly from an outer surface of the body 212. The alignment protrusion 216 is additionally positioned proximate to one receiving feature 214. In some examples, the angular position about the body 212 of the alignment protrusion 216 is aligned with the same angular position of one or more navigation sensors 226 associated with the open distal end 224 of the sleeve 222. Thus, in some examples, the alignment protrusion 216 identifies a longitudinal axis that is common with one or more navigation sensors 226. As will be described in greater detail below, this identification may be beneficial to facilitate insertion of the flexible adapter 200 onto the tissue shaving instrument 100 in a predetermined alignment with correspondence between one or more navigation sensors 226 and the end effector 112. For instance, the operator may ensure that the alignment protrusion 216 is angularly aligned with the first transverse opening 132 about the longitudinal axis of the shaft assembly 120.

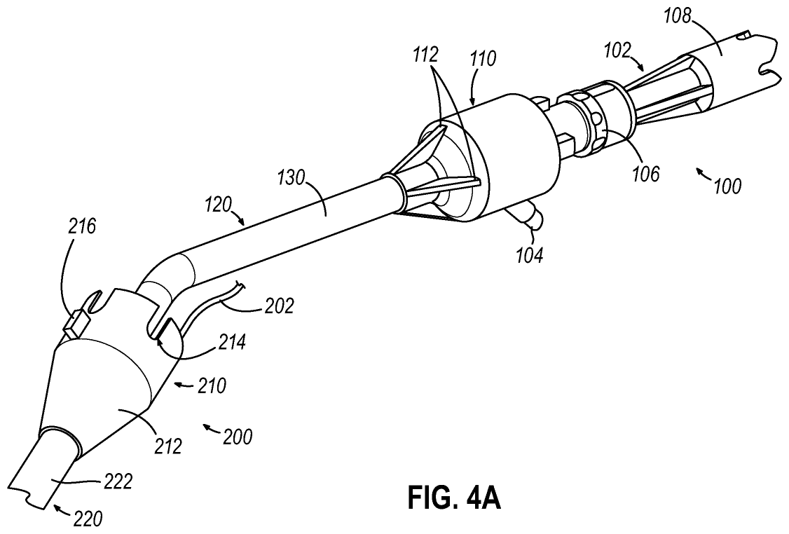

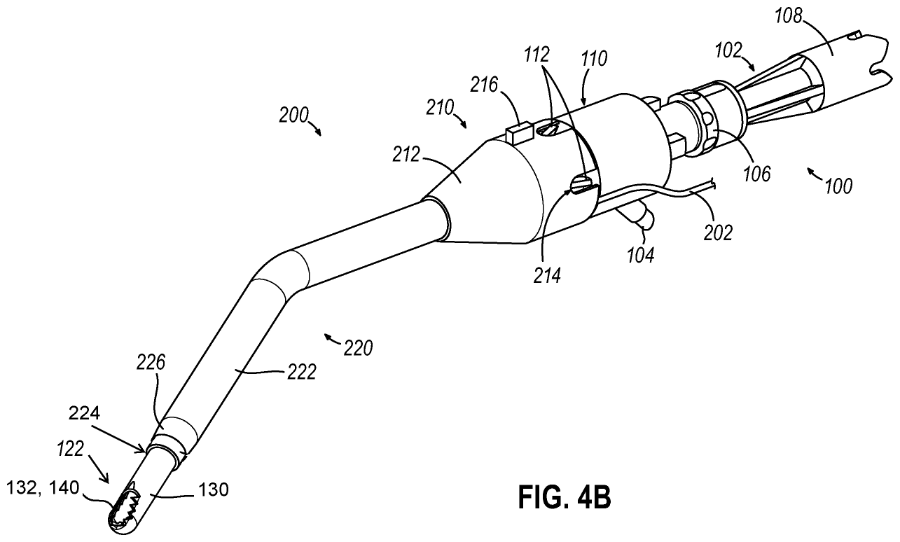

FIGS. 4A and 4B show an example of a use of the flexible adapter 200 in combination with the tissue shaving instrument 100. As best seen in FIG. 4A, the distal end of the shaft assembly 120 of the tissue shaving instrument 100 may initially be inserted into the proximal end of hub 210 of the flexible adapter 200. At this stage, the alignment protrusion 216 of the flexible adapter 200 may be aligned with the end effector 122 of the tissue shaving instrument 100. Such an alignment at this stage is used to ensure alignment between the end effector 122 and one or more navigation sensors 226 once the flexible adapter 200 is fully attached to the tissue shaving instrument 100.

After a desired angular alignment is achieved, the hub 210 of the flexible adapter 200 is moved proximally relative to the tissue shaving instrument 100 along the length of the shaft assembly 120 of the tissue shaving instrument 100 to insert the shaft assembly 120 into the sleeve 222 of the flexible adapter 200. During this insertion, the sleeve 222 is configured to flex or otherwise deform to accommodate the size and shape of the shaft assembly 120 of the tissue shaving instrument 100. For instance, as described above, the shaft assembly 120 may include a bend along its length, while the sleeve 222 of the flexible adapter 200 may initially be straight. Thus, during this insertion, the sleeve 222 may bend to complement a bend defined by the shaft assembly 120. In addition, or in the alternative, some examples of the shaft assembly 120 of the tissue shaving instrument 100 may define a different diameter relative to the diameter of the sleeve 222. Thus, the sleeve 222 may expand in diameter during insertion to accommodate differently sized the shaft assemblies 120 of the tissue shaving instrument 100.

Movement of the hub 210 of the flexible adapter 200 may continue until the hub 210 engages the hub 110 of the tissue shaving instrument 100. At this stage, the ribs 112 of the hub 110 are received within the receiving features 214 of the hub 210, as seen in FIG. 4B. In some examples, this receiving action may function as a poka-yoke feature where the hub 210 may engage the hub 110 in a predetermined angular orientation, such as in one or more predetermined angular orientations. For instance, it may be desirable for one or more navigation sensors 226, or a portion of one or more navigation sensors 226, to have a particular alignment with the end effector 122 (e.g., aligned along a common axis). Such a particular alignment may be desirable to enhance locating the end effector 122, and particularly the first transverse opening 132, in three-dimensional space via one or more navigation sensors 226. Thus, in some embodiments, it may be desirable for the hub 210 to only engage the hub 110 in one or more predetermined ways such as in the position shown where the alignment protrusion 216 is also aligned with the end effector 122 along a common axis. In some examples, each rib 112 and each receiving feature 214 may have a different configuration, size, or shape to further facilitate such a poka-yoke feature. Although such a differing configuration, size, or shape is merely optional and such features may be substantially similar in some examples.

Once the flexible adapter 200 is fully inserted onto the tissue shaving instrument 100, the open distal end 224 of the sleeve 222 is generally positioned proximate to the end effector 122. Additionally, the open distal end 224 and/or other portions of the sleeve 222 are configured to apply a circumferential compression against the shaft assembly 120. Such a circumferential compression is configured to provide sealing engagement between the sleeve 222 and the shaft assembly 120 to prevent fluid ingress into the interior of sleeve 222 and/or one or more portions of the shaft assembly 120. Such a circumferential compression is further configured to compress one or more navigation sensors 226 against the exterior of the shaft assembly 120, thereby holding one or more navigation sensors 226 in position relative to the shaft assembly 120 through friction. It should be understood that in some examples, a portion of the open distal end 224 includes a shaped portion to further facilitate such a circumferential compression.

After the flexible adapter 200 is fully inserted onto the tissue shaving instrument 100, one or more navigation sensors 226 may be used in combination with the IGS navigation system 50 to navigate the tissue shaving instrument 100 via the flexible adapter 200. One or more navigation sensors 226 may be used with the IGS navigation system 50 to navigate the tissue shaving instrument 100 through one or more anatomical passageways of a patient and/or relative to one or more anatomical features of a patient. As described above, such navigation may be combined with navigation of other instruments (e.g., probes) to facilitate real-time overlays of instrument positioning on visualizations. In some uses, such real-time overlays may be used to facilitate true anatomical measurements of patient anatomy, thereby improving procedure workflows and procedure outcomes.

### IV. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

Example 1: An apparatus for use with an instrument, comprising: a hub, the hub being configured to engage a portion of the instrument, in a predetermined angular orientation with respect to an axis of the instrument; a shaft assembly extending from the hub and defining a hollow interior and an open distal end, the hollow interior being configured to receive a tubular portion of the instrument; and one or more navigation sensors, at least one of the one or more navigation sensors being disposed proximate the open distal end of the shaft assembly, the one or more navigation sensors being configured generate signals indicating a position of at least a portion of the shaft assembly in three-dimensional space.

Example 2. The apparatus of Example 1, the one or more navigation sensors including a plurality of navigation sensors.

Example 3. The apparatus of Example 2, the one or more navigation sensors include a first navigation sensor and a second navigation sensor, the first navigation sensor being disposed on the shaft assembly proximate the distal end, the second navigation sensor being disposed on the shaft assembly in a different position relative to the first navigation sensor.

Example 4. The apparatus of any of Examples 2 through 3, the shaft assembly including a sleeve, at least one navigation sensor of the one or more navigation sensors being secured to the sleeve.

Example 5. The apparatus of Example 4, the sleeve being configured to flex to conform to a size and shape of a portion of the instrument.

Example 6. The apparatus of Examples 4 or 5, the sleeve including a shaped portion defining the open distal end of the shaft assembly, the shaped portion of the sleeve being configured to apply circumferential compression against a portion of the instrument.

Example 7. The apparatus of Example 6, at least one navigation sensor of the one or more navigation sensors being secured to the shaped portion, the shaped portion being configured to compress the at least one navigation sensor secured to the shaped portion into a portion of the instrument.

Example 8. The apparatus of any of Examples 1 through 7, a first navigation sensor of the one or more navigation sensors being integrated into or onto the hub.

Example 9. The apparatus of Example 8, a second navigation sensor of the one or more navigation sensors being integrated into a distal end of the shaft assembly.

Example 10. The apparatus of any of Examples 1 through 9, the hub including a plurality of receiving features configured to receive a portion of the instrument.

Example 11. The apparatus of Example 10, each receiving feature defining a slot.

Example 12. The apparatus of Example 11, each slot extending distally from a proximal end of the hub.

Example 13. The apparatus of any of Examples 1 through 12, the hub including an alignment member, the alignment member being positioned on the hub in a predetermined angular orientation corresponding to a predetermined angular orientation of at least one navigation sensor of the one or more navigation sensors.

Example 14. The apparatus of Example 13, the alignment member including an alignment protrusion, the alignment protrusion extending outwardly from an outer surface defined by the hub.

Examples 15. The apparatus of any of Examples 1 through 9, the hub defining an alignment protrusion and a plurality of receiving features, the alignment protrusion being proximate a single receiving feature of the plurality of receiving features.

Example 16. An assembly for use in an ENT procedure, the assembly comprising: a rotary instrument, the rotary instrument including a body, a hub associated with the body, and a shaft extending from a portion of the body or the hub, the shaft including an end effector proximate a distal end of the shaft, the end effector including a rotating feature, the rotating feature being configured to affect tissue; and an adapter, the adapter including: a hub configured to receive a portion of the hub of the tissue shaving instrument at a predetermined angular orientation about a longitudinal axis of the shaft, a shaft assembly extending distally from the hub, the shaft assembly including a sleeve configured to receive the shaft of the tissue shaving instrument within a hollow interior defined by the sleeve, and one or more navigation sensors secured to a portion of the adapter, each navigation sensor being configured to generate signals indicating a position of one or more features of the sleeve in three-dimensional space, at least one navigation sensor of the one or more navigation sensors being proximate the end effector of the tissue shaving instrument when the shaft of the tissue shaving instrument is received within the sleeve of the adapter.

Example 17. The assembly of Example 16, the hub of the tissue shaving instrument including a plurality of locating features, the hub of the adapter including a plurality of receiving features, the receiving features being complementary to the locating features such that each receiving feature is configured to receive a corresponding locating feature.

Example 18. The assembly of Example 17, a single receiving feature of the plurality of receiving features being angularly aligned with an angular position of a navigation sensor of the one or more navigation sensors.

Example 19. The assembly of any of Examples 16 through 18, the hub of the adapter including one or more poka-yoke features, the one or more poka-yoke features being configured to permit engagement between the hub of the tissue shaving instrument and the hub of the adapter in a single predetermined position relative to each other.

Example 20. The assembly of any of Examples 16 through 19, the rotary instrument comprising a tissue shaving instrument

Example 21. A method of using a tissue shaving instrument with a navigation adapter, the method comprising: inserting a shaft of the tissue shaving instrument into a sleeve of the navigation adapter to align an open distal end of the navigation adapter with an end effector of the shaft of the tissue shaving instrument; receiving a portion of the tissue shaving instrument within a portion of the navigation adapter to confirm angular alignment between a navigation sensor coupled to the sleeve of the navigation adapter and the end effector of the tissue shaving instrument; and locating the open distal end of the navigation adapter in three-dimensional space to navigate the end effector of the tissue shaving instrument using the navigation sensor coupled to the sleeve.

Example 22. The method of Example 21, further comprising locating at least one other feature of the navigation adapter in three-dimensional space to navigate one or more elements of the tissue shaving instrument using the navigation sensor.

Example 23. The method of any of Examples 21 through 22, further comprising compressing the navigation sensor onto a surface of the shaft of the tissue shaving instrument using the sleeve of the navigation adapter.

### V. Miscellaneous

It should be understood that any of the teachings, expressions, embodiments, examples, etc. described herein may be combined with any of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present inventions, further adaptations of the methods and systems described herein may be accomplished by appropriate modifications by one skilled in the art without departing from the scope of the present inventions. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present inventions should be considered in terms of the following claims and is understood not to be limited to the details of structure and operation shown and described in the specification and drawings.

What is claimed is:

1. A flexible adapter for use with a tissue shaving instrument, the flexible adapter comprising:
a hub configured to engage a portion of the tissue shaving instrument, in a predetermined angular orientation with respect to an axis of the tissue shaving instrument;
a shaft assembly extending from the hub and defining a hollow interior and an open distal end, the hollow interior being configured to receive a tubular portion of the tissue shaving instrument; and
a navigation sensor being disposed proximate the open distal end of the shaft assembly, the navigation sensor being configured generate a signal indicating a position of at least a portion of the shaft assembly in three-dimensional space.

2. The flexible adapter of claim 1, wherein the navigation sensor comprises a first navigation sensor and a second navigation sensor, the first navigation sensor being disposed on the shaft assembly proximate the distal end, the second navigation sensor being disposed on the shaft assembly in a different position relative to the first navigation sensor.

3. The flexible adapter of claim 1, wherein the shaft assembly comprises a sleeve configured to flex to conform to a size and shape of a portion of the tissue shaving instrument, the navigation sensor being secured to the sleeve.

4. The flexible adapter of claim 3, wherein the sleeve comprises a shaped portion defining the open distal end of the shaft assembly, the shaped portion of the sleeve being configured to apply circumferential compression against a portion of the tissue shaving instrument.

5. The flexible adapter of claim 4, wherein the navigation sensor is secured to the shaped portion, the shaped portion being configured to compress the navigation sensor secured to the shaped portion into a portion of the tissue shaving instrument.

6. The flexible adapter of claim 1, wherein the navigation sensor comprises (i) a first navigation sensor that is integrated into or onto the hub, and (ii) a second navigation sensor that is integrated into a distal end of the shaft assembly.

7. The flexible adapter of claim 1, wherein the hub comprises a plurality of receiving features configured to receive a portion of the tissue shaving instrument.

8. The flexible adapter of claim 7, wherein each receiving feature defines a slot.

9. The flexible adapter of claim 8, wherein each slot extends distally from a proximal end of the hub.

10. The flexible adapter of claim 1, wherein the hub comprises an alignment member, the alignment member being positioned on the hub in a predetermined angular orientation corresponding to a predetermined angular orientation of the navigation sensor.

11. The flexible adapter of claim 10, wherein the alignment member comprises an alignment protrusion, the alignment protrusion extending outwardly from an outer surface defined by the hub.

12. The flexible adapter of claim 1, wherein the hub defines an alignment protrusion and a plurality of receiving features, the alignment protrusion being proximate a single receiving feature of the plurality of receiving features.

13. A navigation assembly for use in an ENT procedure, the navigation assembly comprising:
a rotary instrument, the rotary instrument comprising a body, a rotary hub associated with the body, and a shaft extending from a portion of the body or the rotary hub, wherein the shaft comprises an end effector proximate a distal end of the shaft, wherein the end effector comprises a rotating feature configured to affect tissue; and
an adapter, the adapter comprising:
an adapter hub configured to receive a portion of the rotary hub at only a predetermined angular orientation about a longitudinal axis of the shaft,
a shaft assembly extending distally from the adapter hub, the shaft assembly comprising a sleeve configured to receive the shaft of the tissue shaving instrument within a hollow interior defined by the sleeve, and
a navigation sensor secured to a portion of the adapter and being configured to generate a signal indicating a position of one or more features of the sleeve in three-dimensional space, the navigation sensor being positionable proximate the end effector of the tissue shaving instrument when the shaft of the tissue shaving instrument is received within the sleeve of the adapter.

14. The navigation assembly of claim 13, wherein the rotary hub comprises a plurality of locating features, the adapter hub comprises a plurality of receiving features, wherein the receiving features are complementary to the locating features such that each receiving feature is configured to receive a corresponding locating feature.

15. The navigation assembly of claim 14, wherein a single receiving feature of the plurality of receiving features is angularly aligned with an angular position of the navigation sensor.

16. The navigation assembly of claim 13, wherein the adapter hub comprises one or more poka-yoke features configured to permit engagement between the rotary hub and the adapter hub in a single predetermined position relative to each other.

17. The navigation assembly of claim 13, wherein the rotary instrument comprises a tissue shaving instrument.

18. A method of using a tissue shaving instrument with a navigation adapter, the method comprising:
inserting a shaft of the tissue shaving instrument into a sleeve of the navigation adapter to align an open distal end of the navigation adapter with an end effector of the shaft of the tissue shaving instrument;
receiving a portion of the tissue shaving instrument within a portion of the navigation adapter to confirm angular alignment between a navigation sensor coupled to the sleeve of the navigation adapter and the end effector of the tissue shaving instrument; and
locating the open distal end of the navigation adapter in three-dimensional space to navigate the end effector of the tissue shaving instrument using the navigation sensor coupled to the sleeve.

19. The method of claim 18, further comprising locating at least one other feature of the navigation adapter in three-dimensional space to navigate one or more elements of the tissue shaving instrument using the navigation sensor.

20. The method of claim 18, further comprising compressing the navigation sensor onto a surface of the shaft of the tissue shaving instrument using the sleeve of the navigation adapter.
